(19) United States
(12) Patent Application Publication
(10) Pub. No.: US 2009/0050142 A1
(43) Pub. Date: Feb. 26, 2009

(54) INHALER DEVICE

(75) Inventor: SOJI HAMANO, Yokohama-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/184,645

(22) Filed: Aug. 1, 2008

(30) Foreign Application Priority Data
Aug. 23, 2007 (JP) 2007-216874

(51) Int. Cl. A61M 11/00

(52) U.S. Cl. 128/200.23

(57) ABSTRACT

A medicine cartridge that discharges medicine towards an air channel includes a liquid medicine tank, a liquid medicine channel, and a discharge head. Before inhalation, the discharge head can be filled with the medicine by pushing a rubber stopper, provided at the bottom of the liquid medicine tank, using a rubber-stopper pushing portion. After a user inhales the medicine, a door for an air inlet is closed and a mouthpiece is slid into the air channel, causing the air pressure in the air channel to increase. This allows the medicine remaining in the discharge head to be returned to the liquid medicine tank.

### BACKGROUND OF THE INVENTION

1. Field of the Invention

The present invention relates to an inhaler device configured to discharge medicine in the form of fine droplets to be inhaled by a user.

2. Description of the Related Art

Inhaler devices can be used by users to ingest medicine by inhalation through the devices. With the use of these inhaler devices and information databases such as electronic medical charts, treatments for the users can be properly implemented. An inhaler device is also a portable terminal that functions both as a storage unit for storing personal information about the user, which includes information about the user's medical chart and prescription, and as a sprayer for spraying the medicine to allow the user to inhale the medicine. An inhaler device also has a spray controller that controls the discharging of the medicine from the sprayer based on the user's inhalation profile so that the user can inhale the medicine in accordance with the information about the prescription. International Publications Nos. WO95/01137 and WO02/04043 disclose examples of such inhaler devices.

In such a medicine inhaler device, a discharge head is used for discharging liquid medicine to be inhaled by the user in the form of fine liquid droplets. For the inhalation, it is desirable in terms of simple manipulation that, for example, one medicine tank has the capacity to hold enough insulin for consecutive administrations three times a day (i.e., morning, noon, and evening) over a seven-day period. If the same discharge head is used for multiple inhalations to fulfill this condition, the liquid medicine would be left in the liquid chambers of the discharge head when the device is not in use. This can lead to the following problems.

Specifically, when the device is not in use, the liquid medicine left in and near the nozzles of the discharge heads may dry out, causing the components of the liquid medicine to become deposited within the discharge head. These deposited components may block the nozzles at the time of the subsequent discharge process, thus unfavorably reducing the discharged amount of medicine. Since the user needs to inhale a specific amount of medicine predetermined based on prescription, such a reduction in the discharged amount is undesirable.

### SUMMARY OF THE INVENTION

The present invention provides an inhaler device that can reduce the time that the elements included in a discharge head serving as a medicine discharger are in contact with various kinds of liquid medicine (medicine) used, so as to avoid discharge defects caused by deposition of medicinal components in the discharge head.

According to an aspect of the invention, an inhaler device includes a medicine container configured to contain medicine; a medicine discharger configured to discharge the medicine supplied from the medicine container; and a pressurizing unit configured to increase a pressure applied to the medicine discharger so as to return the medicine in the medicine discharger to the medicine container.

Accordingly, the medicine remaining in the medicine discharger after a discharge process can be automatically returned to the medicine container. As a result, the contact time between the medicine and the elements included in the medicine discharger can advantageously be reduced, thereby preventing the medicine from becoming deposited in the medicine discharger.

Further features of the present invention will become apparent from the following description of exemplary embodiments with reference to the attached drawings, in which like reference characters designate the same or similar parts throughout the figures.

### BRIEF DESCRIPTION OF THE DRAWINGS

FIG. 1A is an external perspective view of an inhaler device according to an exemplary embodiment of the present invention, in which an access cover thereof is shown in a closed state; and FIG. 1B is an external perspective view of the inhaler device, in which the access cover thereof is shown in an open state.

FIG. 2 is a schematic cross-sectional view of a medicine cartridge according to an exemplary embodiment of the present invention.

FIG. 3A is a schematic cross-sectional view showing an internal structure of an inhaler device according to a first embodiment of the present invention; and FIG. 3B is a block diagram showing an electrical configuration of the inhaler device according to the first embodiment.

FIG. 4 is a flow chart of an operation according to the first embodiment.

FIG. 5 is a graph that shows a pressure change in an air channel.

FIG. 6A is a schematic cross-sectional view showing an internal structure of an inhaler device according to a second embodiment of the present invention; and FIG. 6B is a block diagram showing an electrical configuration of the inhaler device according to the second embodiment.

FIG. 7 is a flow chart of an operation according to the second embodiment.

FIG. 8A is a schematic cross-sectional view showing an internal structure of an inhaler device according to a third embodiment of the present invention; and FIG. 8B is a block diagram showing an electrical configuration of the inhaler device according to the third embodiment.

FIG. 9A is a schematic cross-sectional view showing a positional relationship between an air channel and a cover in the third embodiment, as viewed from above the inhaler device; and FIG. 9B is a schematic cross-sectional view also showing the positional relationship between the air channel and the cover in the third embodiment, when the inhaler device is viewed laterally.

FIG. 10 is a flow chart of an operation according to the third embodiment.

### DESCRIPTION OF THE EMBODIMENTS

The preferred embodiments of the present invention will now be described in detail in accordance with the accompanying drawings.

An inhaler device according to an exemplary embodiment of the present invention is provided with a pressurizing unit configured to increase the pressure applied to a discharge head serving as a medicine discharger, so as to return the medicine remaining in the medicine discharger after a discharge process to a liquid medicine tank serving as a medicine container. The medicine can then be preserved in the liquid medicine tank. This permits maintenance of a state in which there is no medicine remaining in the discharge head when the device is not in use, and can thus prevent discharge defects caused by deposition of medicinal components. (Herein, the medicine discharger is sometimes also called a “medicine ejector”.)

An exemplary embodiment of the present invention will now be described with reference to the drawings.

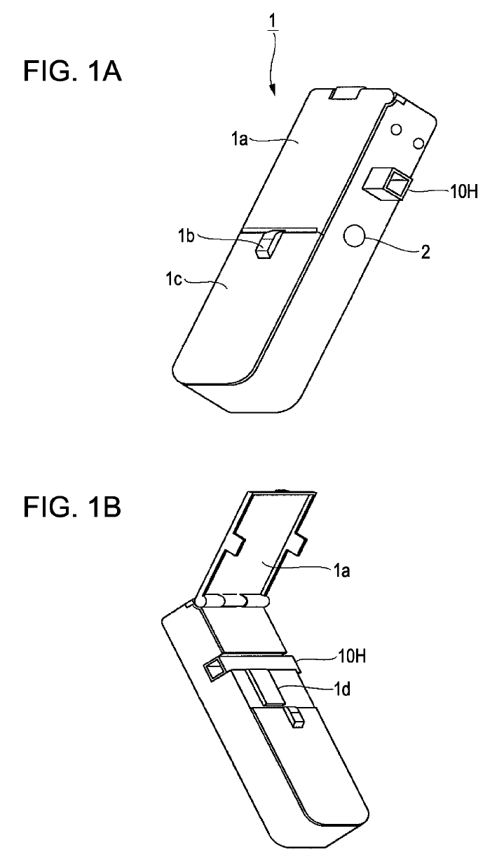

FIG. 1A is an external perspective view of an inhaler device 1 according to an exemplary embodiment of the present invention. FIG. 1B is a perspective view of the inhaler device 1, in which an access cover 1a thereof is shown in an open state. When loading or unloading a medicine cartridge 1d, the access cover 1a can be opened and closed using a lock lever 1b.

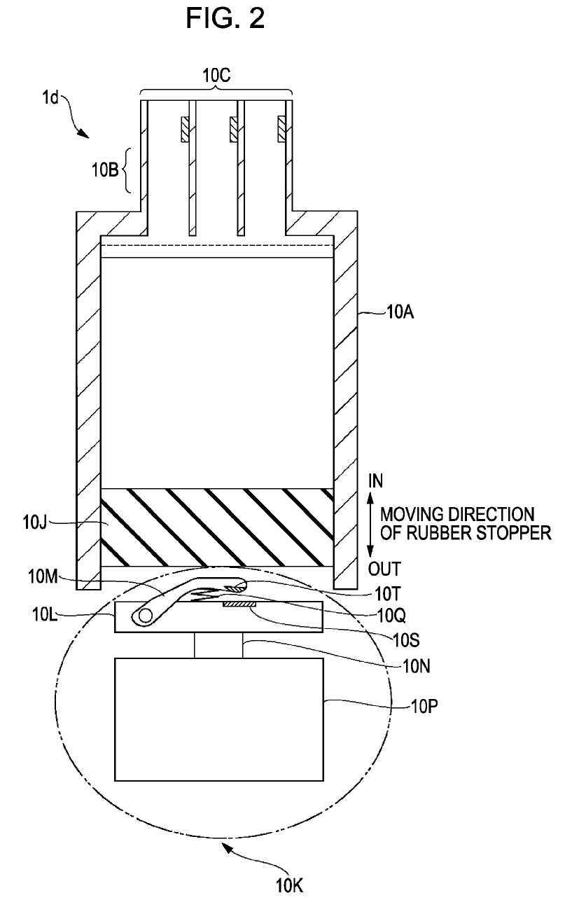

First, a user may hold the inhaler device 1, release the lock lever 1b disposed on a front cover 1c, and open the access cover 1a. Then, referring to FIG. 2, the user may load the medicine cartridge 1d, which integrally includes a liquid medicine tank 10A, a liquid medicine channel 10B, and a discharge head 10C serving as a medicine discharger, and close the access cover 1a. Subsequently, the user may press a power switch 2 to turn on the power. The user may then exhale deeply, put a mouthpiece 10H into the mouth, and inhale deeply and slowly. The inhaler device 1 has a sensor for detecting the start of inhalation by the user, and discharges a predetermined amount of medicine in the form of liquid droplets having a diameter of about 3 μm for 1 second immediately upon the detection. The discharged medicine flows through the user's mouth and throat and then into the lungs along with the flow of air inhaled by the user.

Having performed this slow deep inhalation, the user holds his/her breath for about 5 to 6 seconds. The liquid droplets of medicine that have reached the lungs adhere inside the lungs. The user may then exhale and resume normal breathing. The inhaler device 1 automatically turns off its power. On the other hand, the medicine adhered to the lungs passes into the bloodstream and runs throughout the user's body, whereby the medicine starts to take effect.

FIG. 2 is a schematic cross-sectional view showing the internal structure of the medicine cartridge 1d.

The medicine cartridge 1d has an integrated structure that includes the liquid medicine tank 10A serving as a medicine container, the liquid medicine channel 10B, the discharge head 10C serving as a medicine discharger, and a rubber stopper 10J. Frictional force between the liquid medicine tank 10A and the rubber stopper 10J is adjusted such that when the difference in pressure between the air outside the rubber stopper 10J and the liquid medicine in the liquid medicine tank 10A becomes ±0.8 kPa or higher, the rubber stopper 10J moves in a direction shown with an arrow (sometimes referred to herein as a “rubber-stopper moving direction”). Specifically, when the external pressure is lower than the internal pressure, the rubber stopper 10J moves outward to increase the capacity of the liquid medicine tank 10A. In contrast, when the internal pressure is lower than the external pressure, the rubber stopper 10J moves inward to reduce the capacity of the liquid medicine tank 10A. To fill the discharge head 10C with the liquid medicine from the liquid medicine tank 10A just before inhalation, the user may push a rubber-stopper pushing portion 10K by a certain amount to move the rubber stopper 10J.

Before this filling process, the rubber stopper 10J is kept distant from a pushing section 10L and a rubber-stopper contact detecting section 10M of the rubber-stopper pushing portion 10K by a predetermined distance or more. When the filling process is to be performed, a pressure-force control section 10P performs control to extend a pressing-force transmission section 10N so as to bring the rubber-stopper contact detecting section 10M on the pushing section 10L into contact with the rubber stopper 10J. While the rubber-stopper contact detecting section 10M is in contact with the rubber stopper 10J, the pressing-force transmission section 10N is further extended, causing a spring 10Q to become compressed. As a result, detection terminals 10S and 10T come into contact with each other. Accordingly, the pressure-force control section 10P can detect that the rubber-stopper contact detecting section 10M is in contact with the rubber stopper 10J.

Subsequently, the pressure-force control section 10P controls the pushing section 10L through the pressing-force transmission section 10N for a predetermined number of steps so as to push the rubber stopper 10J further by a predetermined distance. Finally, the pressure-force control section 10P performs control to return the pressing-force transmission section 10N to its original position by pulling the pressing-force transmission section 10N by a distance the pressing-force transmission section 10N was extended (i.e., the total distance the pressing-force transmission section 10N was moved in the contact operation and the pushing operation). This completes the operation.

In other words, the rubber-stopper pushing portion 10K is configured to be used for pushing the rubber stopper 10J only at the time of the filling process, but other than that, the rubber-stopper pushing portion 10K is maintained at a fixed position distant from the rubber stopper 10J. With the rubber-stopper pushing portion 10K being positioned distant from the rubber stopper 10J in this manner, the rubber stopper 10J can move within the liquid medicine tank 10A in accordance with the difference in pressure described above.

In this exemplary embodiment of the present invention, the medicine discharger (discharge head 10C) may have a desired discharge-energy generating element. Examples of a discharge-energy generating element include an electrothermal transducer configured to apply thermal energy to the medicine, and an electromechanical transducer configured to apply mechanical energy to the medicine. In other words, one example of a medicine discharge method is a thermal jet method in which an electrothermal transducer is used to apply thermal energy to the medicine for discharging the medicine from nozzles. Another example is a method that employs an electromechanical transducer (such as a piezoelectric element) that can apply mechanical energy to the medicine. In this method, the medicine is discharged from nozzles by utilizing vibratory pressure of the electromechanical transducer. An appropriate discharge method may be selected in accordance with the kind of medicine used.

Employing a thermal jet method can allow for enhancement in each of the medicine discharge units in terms of the nozzle aperture, the quantity of heat in a thermal pulse used during the discharging, the dimensional accuracy of a micro-heater used as an electrothermal transducer, and reproducibility. Therefore, a narrow droplet-diameter distribution can be achieved. In addition, a thermal jet method has high applicability to small devices that contain low-production-cost discharge heads and that require frequent replacement of the discharge heads. Therefore, if a user demands portability and user-friendliness in a medicine discharge device, a discharge device employing a thermal jet method is particularly suitable.

The liquid medicine used in the present invention refers to a medicine in a liquid state or a liquid medium containing medicine, and is not limited to a medicinal compound that exhibits pharmacological or physiological effects. In addition to such medicinal compound, the liquid medicine may contain a flavoring agent, an aroma-providing component, a colorant, a pigment, etc. Moreover, the liquid medicine may contain a desired additive or additives. The medicine in the liquid may be in a dissolved state, dispersed state, emulsified state, suspension state, or slurry state, and it is preferable that the medicine is distributed evenly throughout the liquid.

When using liquid medicine as the medicine, the main medium of the liquid is preferably water or an organic material. In view of the fact that the medicine will enter a living body, it is preferable that water be used as the main medium.

### First Embodiment

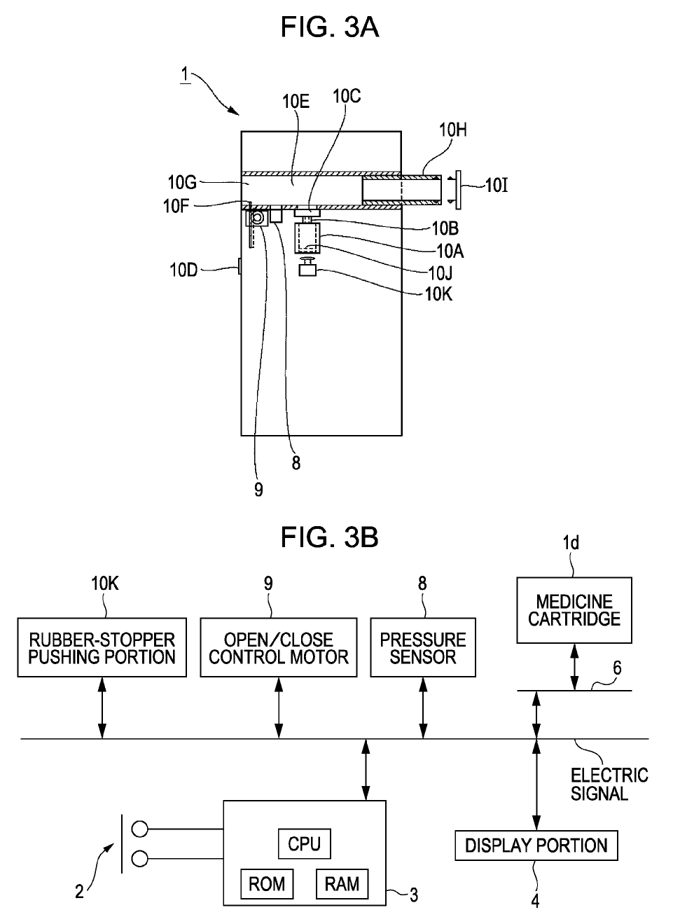

FIG. 3A is a schematic cross-sectional view of an inhaler device 1 according to a first embodiment of the present invention. A main body of the inhaler device 1 has disposed therein an air hole 10D, an air channel 10E, a door 10F, an air inlet 10G, and an inhaler portion including, for example, a mouthpiece 10H. A pressure sensor 8 is configured to detect a difference in air pressure between the interior of the air channel 10E and the outside. The pressure sensor 8 can detect a storing operation of the mouthpiece 10H on the basis of a pressure change. An open/close control motor 9 is configured to perform control to close the door 10F prior to the storing operation of the mouthpiece 10H.

A liquid medicine tank 10A is configured to supply liquid medicine to a discharge head 10C through a liquid medicine channel 10B. The liquid medicine channel 10B serves as a channel through which the liquid medicine can be supplied from the liquid medicine tank 10A to the discharge head 10C. The discharge head 10C serves as a discharge unit configured to discharge the liquid medicine towards the air channel 10E. The main body of the inhaler device 1 has an air hole 10D for permitting the ambient air pressure to be detected by the pressure sensor 8 so that the pressure sensor 8 can determine the difference in air pressure between the outside and the air channel 10E. The air channel 10E is configured to mix liquid droplets discharged from the discharge head 10C with air flowing in through the air inlet 10G and to guide the mixture towards the mouthpiece 10H. The discharge head 10C serving as a medicine discharger is disposed facing the air channel 10E so that the liquid medicine can be discharged to the air channel 10E. The mouthpiece 10H is slidable relative to the air channel 10E and is thus storable inside the inhaler device 1. For inhalation, the user can pull out the mouthpiece 10H and put the mouthpiece 10H into the mouth.

Before inhalation, the user may slide the mouthpiece 10H relative to the air channel 10E until the mouthpiece 10H is fully extended. Subsequently, a mouthpiece cap 10I is removed. The user may then put the mouthpiece 10H into the mouth and start the inhalation process.

When the inhalation process is finished, the user may attach the mouthpiece cap 10I onto the mouthpiece 10H. One of the purposes for attaching the mouthpiece cap 10I onto the mouthpiece 10H is to prevent the air channel 10E from becoming contaminated. The storable mouthpiece 10H is then inserted and stored in the main body of the inhaler device 1. At the same time, the open/close control motor 9 controls the door 10F to close the air inlet 10G. Consequently, the pressure in the air channel 10E increases by an amount corresponding to the inserted volume of the storable mouthpiece 10H serving as a pressurizing unit, whereby the pressure in the air channel 10E becomes higher than the ambient air pressure. This implies that the pressure applied to the discharge head 10C increases. On the other hand, the liquid medicine tank 10A has its bottom sealed with a rubber stopper 10J to prevent leakage of the liquid medicine. As mentioned above, the rubber stopper 10J is configured to move within the liquid medicine tank 10A when the difference in pressure between the air outside the rubber stopper 10J and the liquid medicine in the liquid medicine tank 10A becomes 0.8 kPa or higher. The rubber stopper 10J is configured to receive the ambient air pressure through the air hole 10D. Therefore, when the mouthpiece 10H is inserted and stored in the main body of the inhaler device 1 and the air pressure in the air channel 10E continues to be at least 0.8 kPa higher than the ambient air pressure for a predetermined time, the rubber stopper 10J moves by a distance corresponding to an amount of liquid medicine withdrawn from the discharge head 10C to the liquid medicine tank 10A. In this manner, the liquid medicine remaining in the discharge head 10C can be returned to the liquid medicine tank 10A after each use, whereby the liquid chambers in the discharge head 10C can be kept free of liquid medicine when the liquid medicine is in a preserved state.

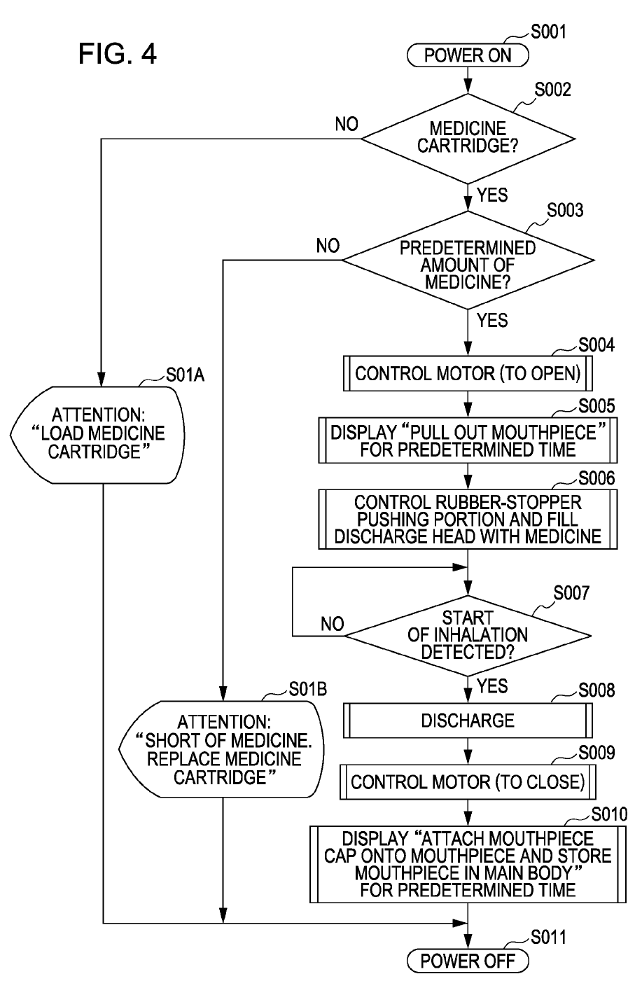

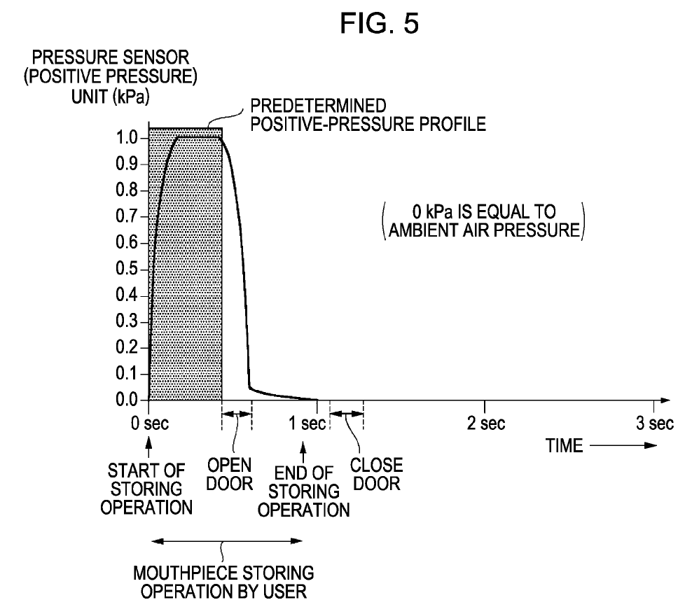

FIG. 3B is a block diagram showing an electrical configuration of the inhaler device 1 according to the first embodiment. FIG. 4 is a flow chart illustrating an operation executed by a control unit 3. FIG. 5 is a graph that shows a change in air-pressure difference between the air channel 10E and the outside that occurs as the mouthpiece 10H with the mouthpiece cap 10I attached thereto is inserted into the main body of the inhaler device 1.

An operation according to the first embodiment will be described in detail below with reference to these drawings.

First, when the power switch 2 is pressed, the control unit 3 turns on the power of the inhaler device 1 in step S001. In step S002, the control unit 3 checks whether the medicine cartridge (i.e. medicine discharger) 1d integrally including the discharge head 10C, the liquid medicine channel 10B, the liquid medicine tank 10A, and the rubber stopper 10J is loaded in the inhaler device 1 and electrically connected with an electric connection component 6 on the basis of an electric signal. If “no”, the control unit 3 displays the message “LOAD MEDICINE CARTRIDGE” on a display portion 4 for a predetermined time in step S01A and subsequently turns off the power of the inhaler device 1 in step S011 to end the operation.

If “yes” in step S002, the control unit 3 determines in step S003 whether the medicine cartridge 1d contains at least a predetermined amount of medicine (for one inhalation process) on the basis of an electric signal from the medicine cartridge 1d. If “no” in step S003, the control unit 3 displays the message “SHORT OF MEDICINE. REPLACE MEDICINE CARTRIDGE” on the display portion 4 for a predetermined time in step S01B and subsequently turns off the power of the inhaler device 1 in step S011 to end the operation.

If “yes” in step S003, the control unit 3 controls the open/close control motor 9 for the air inlet 10G in step S004 so as to fully open the air inlet 10G. In step S005, the control unit 3 displays the message “PULL OUT MOUTHPIECE” on the display portion 4 for a predetermined time.

In step S006, the control unit 3 controls the rubber-stopper pushing portion 10K so as to fill the discharge head 10C with the liquid medicine.

In step S007, the control unit 3 detects the difference in air pressure between the outside and the air channel 10E through the pressure sensor 8. The control unit 3 can detect the start of inhalation by the user when the air-pressure difference reaches a predetermined value. In step S008, a predetermined amount (20 μL) of liquid medicine is discharged for a predetermined time (1 second). After the user completes the inhalation, the control unit 3 controls the open/close control motor 9, which is configured to open and close the door 10F for the air inlet 10G, in step S009 so as to completely close the air inlet 10G of the air channel 10E. In step S010, the control unit 3 displays the message “ATTACH MOUTHPIECE CAP 10I ONTO MOUTHPIECE 10H AND STORE MOUTHPIECE 10H IN MAIN BODY” on the display portion 4 for a predetermined time (5 seconds). While the message is being displayed for the predetermined time, the control unit 3 monitors a value detected by the pressure sensor 8.

Referring to FIG. 5, if a positive-pressure state where the pressure in the air channel 10E is at least 0.8 kPa higher than the ambient air pressure (that is, a state where the pressure in the air channel 10E is higher than the ambient air pressure) continues for a predetermined time, the control unit 3 determines that the liquid medicine has been withdrawn from the discharge head 10C to the liquid medicine tank 10A (that is, there is no liquid medicine remaining in the discharge head 10C). The control unit 3 then controls the open/close control motor 9 for the air inlet 10G so as to fully open the air inlet 10G. After a lapse of 0.5 seconds, the control unit 3 determines that the storing operation of the mouthpiece 10H is complete, and controls the open/close control motor 9 for the air inlet 10G again so as to completely close the air inlet 10G. With the mouthpiece cap 10I attached to the mouthpiece 10H and the air inlet 10G in a completely closed state, the inhaler device 1 can be kept free of foreign matter when not in use. Finally, in step S011, the control unit 3 turns off the power of the inhaler device 1 so as to end the operation.

### Second Embodiment

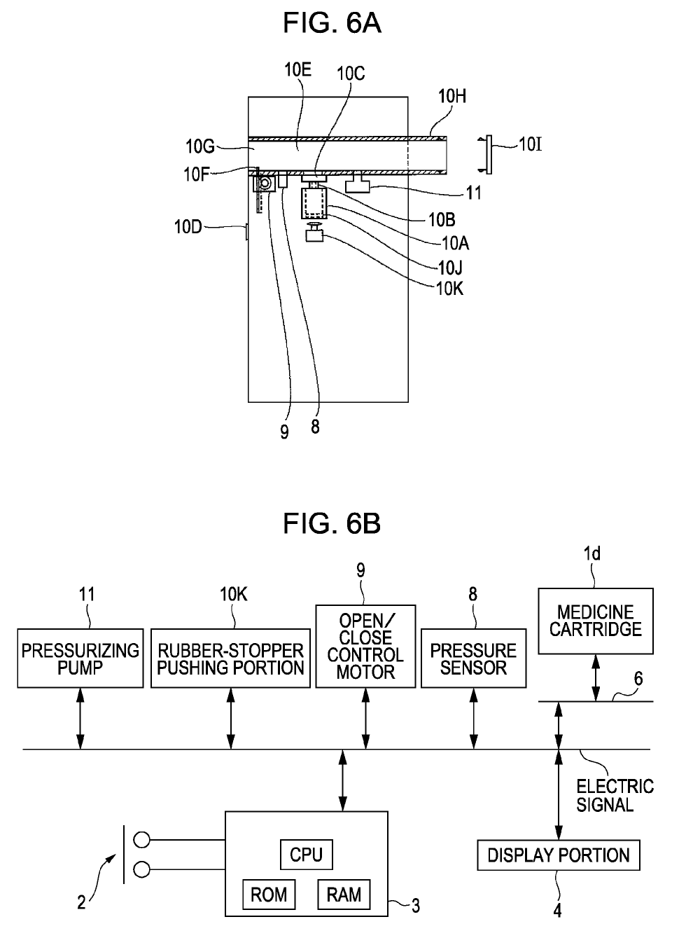

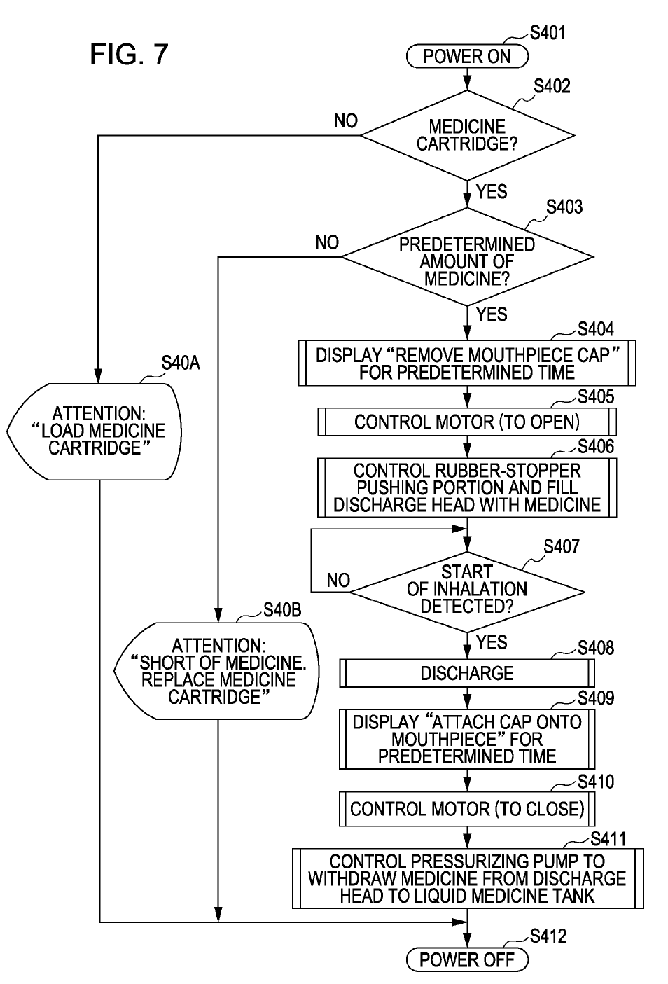

FIGS. 6A, 6B, and 7 are diagrams for explaining an inhaler device 1 according to a second embodiment of the present invention.

In contrast to the first embodiment in which the pressure in the air channel 10E is increased by sliding the mouthpiece 10H within the air channel 10E, the second embodiment is equipped with a pressurizing pump (pressurizing unit) 11 for increasing the pressure in the air channel 10E. Other than that, the second embodiment is similar to the first embodiment, in having the pressure sensor 8 that detects the difference in air pressure between the air channel 10E and the outside and the open/close control motor 9 that controls the opening and closing of the door 10F. In addition, the second embodiment is also provided with the medicine cartridge 1d that includes, for example, the liquid medicine tank 10A configured to supply liquid medicine to the discharge head 10C through the liquid medicine channel 10B, and the liquid medicine channel 10B through which the liquid medicine is supplied from the liquid medicine tank 10A to the discharge head 10C. The main body of the inhaler device 1 has the air hole 10D for sending an ambient air pressure to the pressure sensor 8 so that the pressure sensor 8 can determine the difference in air pressure between the outside and the air channel 10E.

Liquid droplets discharged from the discharge head 10C to the air channel 10E are mixed with air flowing in through the air inlet 10G. The mixture is then delivered into the mouth of a human body through the mouthpiece 10H. To keep the air channel 10E free of foreign matter when the inhaler device 1 is in a stored state and not being used by the user for inhalation, the mouthpiece cap 10I may be attached to the mouthpiece 10H.

FIG. 6B is a block diagram showing an electrical configuration of the inhaler device 1 according to the second embodiment. FIG. 7 is a flow chart illustrating an operation executed by the control unit 3 in the second embodiment.

An operation according to the second embodiment will be described in detail below with reference to these drawings.

First, when the power switch 2 is pressed, the control unit 3 turns on the power of the inhaler device 1 in step S401. In step S402, the control unit 3 checks whether the medicine cartridge 1d integrally including the discharge head 10C, the liquid medicine channel 10B, the liquid medicine tank 10A, and the rubber stopper 10J is loaded in the inhaler device 1 on the basis of an electric signal. If “no”, the control unit 3 displays the message “LOAD MEDICINE CARTRIDGE” on the display portion 4 for a predetermined time in step S40A and subsequently turns off the power of the inhaler device 1 in step S412 to end the operation.

If “yes” in step S402, the control unit 3 determines in step S403 whether the medicine cartridge 1d contains at least a predetermined amount of medicine (for one inhalation process) on the basis of an electric signal from the medicine cartridge 1d. If “no” in step S403, the control unit 3 displays the message “SHORT OF MEDICINE. REPLACE MEDICINE CARTRIDGE” on the display portion 4 for a predetermined time in step S40B and subsequently turns off the power of the inhaler device 1 in step S412 to end the operation.

If “yes” in step S403, the control unit 3 displays the message “REMOVE MOUTHPIECE CAP” on the display portion 4 for a predetermined time in step S404. In this case, the user may remove the mouthpiece cap 10I. In step S405, the control unit 3 controls the open/close control motor 9 so as to fully open the air inlet 10G.

In step S406, the control unit 3 controls the rubber-stopper pushing portion 10K so as to fill the discharge head 10C with the liquid medicine. The rubber-stopper pushing portion 10K operates in the same manner as in the first embodiment.

In step S407, the control unit 3 detects the difference in air pressure between the outside and the air channel 10E through the pressure sensor 8. The control unit 3 can detect the start of inhalation by the user when the air-pressure difference reaches a predetermined value.

In step S408, a predetermined amount (20 μL) of liquid medicine is discharged for a predetermined time (1 second). In step S409, the control unit 3 displays the message “ATTACH CAP ONTO MOUTHPIECE” on the display portion 4 for a predetermined time. In step S410, the control unit 3 controls the open/close control motor 9 for the air inlet 10G so as to completely close the air inlet 10G of the air channel 10E. In step S411, the control unit 3 controls the pressurizing pump 11 so as to withdraw the liquid medicine from the discharge head 10C to the liquid medicine tank 10A through the liquid medicine channel 10B. In this case, since the control unit 3 is controlling the open/close control motor 9 to close the air inlet 10G, the pressure in the air channel 10E becomes higher than the ambient air pressure by an amount the pressurizing pump 11 is controlled for withdrawing the liquid medicine from the discharge head 10C to the liquid medicine tank 10A through the liquid medicine channel 10B. This implies that the pressure applied to the discharge head 10C increases.

On the other hand, the liquid medicine tank 10A has the rubber stopper 10J attached thereto, which is configured to move within the liquid medicine tank 10A when the difference in pressure between the air outside the rubber stopper 10J and the liquid medicine in the liquid medicine tank 10A becomes 0.8 kPa or higher. Therefore, when the pressure difference is kept at 0.8 kPa or higher for a predetermined time by controlling the pressurizing pump 11, the rubber stopper 10J moves by a distance corresponding to an amount of liquid medicine withdrawn from the discharge head 10C to the liquid medicine tank 10A. Finally, in step S412, the control unit 3 turns off the power of the inhaler device 1 so as to end the operation. In this manner, the liquid medicine remaining in the discharge head 10C can be returned to the liquid medicine tank 10A after each use, whereby the liquid chambers in the discharge head 10C can be kept free of liquid medicine when the liquid medicine is in a preserved state.

### Third Embodiment

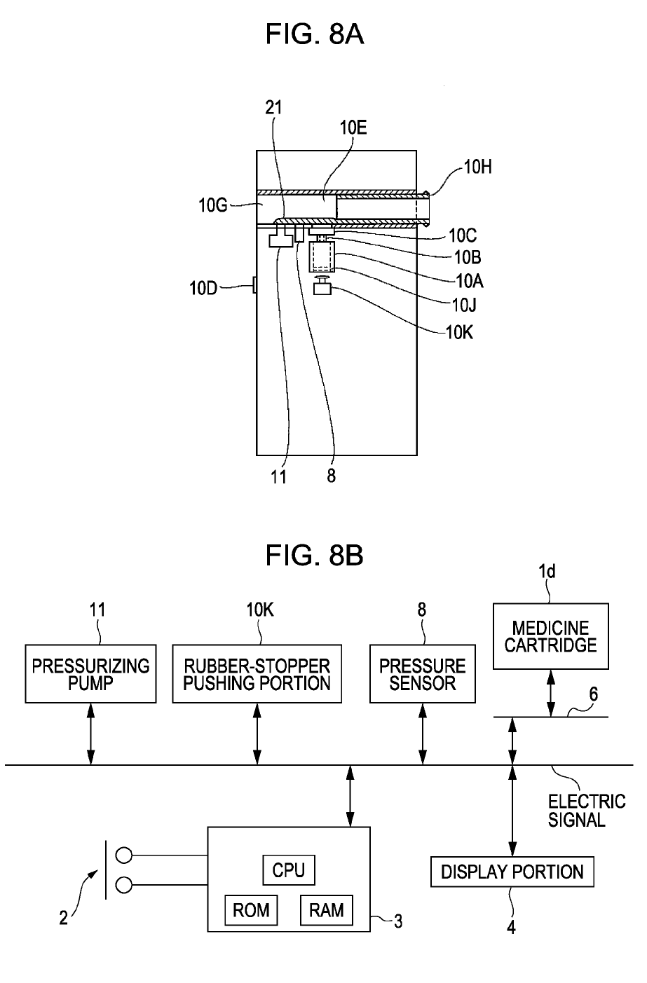

FIGS. 8A to 10 are diagrams for explaining an inhaler device 1 according to a third embodiment of the present invention.

Referring to FIG. 8A, the third embodiment is equipped with a cover 21 that is integrated with the mouthpiece 10H slidable within the air channel 10E, and is also equipped with the pressurizing pump (pressurizing unit) 11. The third embodiment is similar to the first embodiment, in having the pressure sensor 8 that detects the difference in air pressure between the air channel 10E and the outside, but differs from the first embodiment in that the door 10F and the open/close control motor 9 have been omitted. The cover 21 is capable of sealing holes (openings) through which the discharge head 10C, the pressure sensor 8, and the pressurizing pump 11 communicate with the air channel 10E.

The main body of the inhaler device 1 has the air hole 10D for sending an ambient air pressure to the pressure sensor 8 so that the pressure sensor 8 can determine the difference in air pressure between the outside and the air channel 10E. Liquid droplets discharged from the discharge head 10C are mixed with air flowing into the air channel 10E through the air inlet 10G. The mixture is then delivered into the mouth of a human body through the mouthpiece 10H.

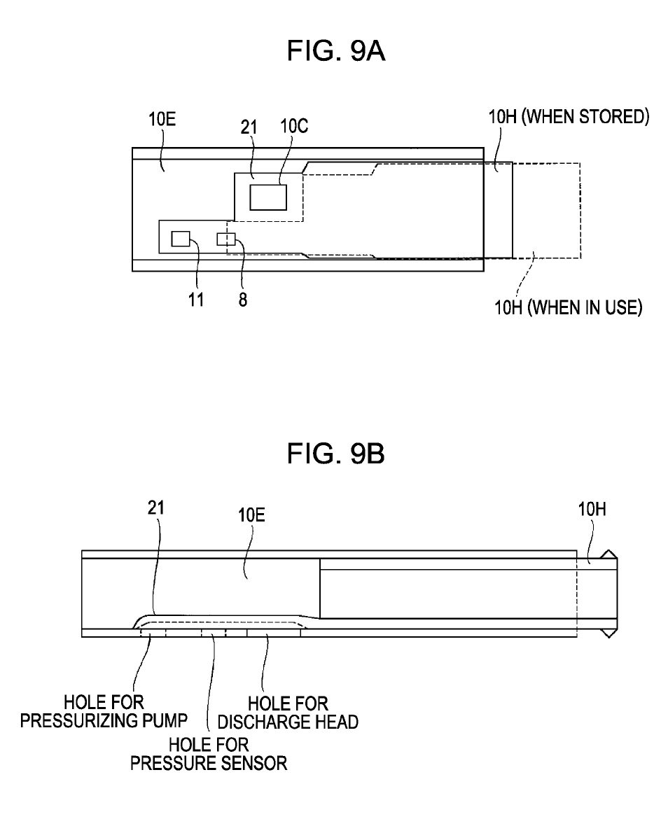

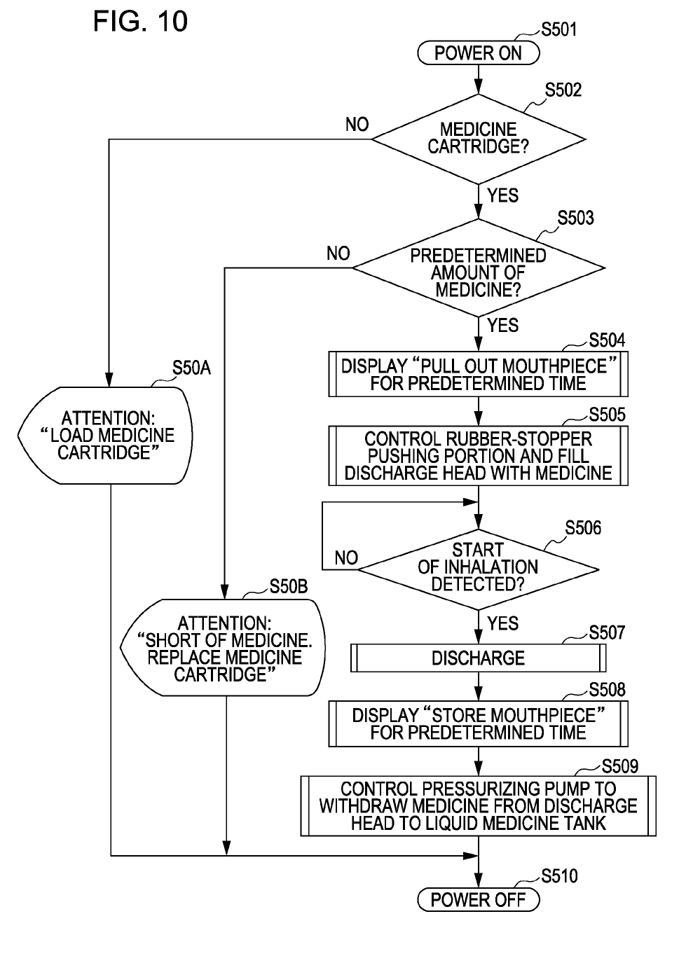

FIG. 8B is a block diagram showing an electrical configuration of the inhaler device 1 according to the third embodiment. FIGS. 9A and 9B illustrate the operation of the cover 21. FIG. 10 is a flow chart illustrating an operation executed by the control unit 3 in the third embodiment.

First, when the power switch 2 is pressed, the control unit 3 turns on the power of the inhaler device 1 in step S501. In step S502, the control unit 3 checks whether the medicine cartridge 1d integrally including the discharge head 10C, the liquid medicine channel 10B, the liquid medicine tank 10A, and the rubber stopper 10J is loaded in the inhaler device 1 on the basis of an electric signal. If “no”, the control unit 3 displays the message “LOAD MEDICINE CARTRIDGE” on the display portion 4 for a predetermined time in step S50A and subsequently turns off the power of the inhaler device 1 in step S510 to end the operation.

If “yes” in step S502, the control unit 3 determines in step S503 whether the medicine cartridge 1d contains at least a predetermined amount of medicine (for one inhalation process) on the basis of an electric signal from the medicine cartridge 1d. If “no” in step S503, the control unit 3 displays the message “SHORT OF MEDICINE. REPLACE MEDICINE CARTRIDGE” on the display portion 4 for a predetermined time in step S50B and subsequently turns off the power of the inhaler device 1 in step S510 to end the operation.

If “yes” in step S503, the control unit 3 displays the message “PULL OUT MOUTHPIECE” on the display portion 4 for a predetermined time in step S504.

In step S505, the control unit 3 controls the rubber-stopper pushing portion 10K so as to fill the discharge head 10C with the liquid medicine. The rubber-stopper pushing portion 10K operates in the same manner as in the first embodiment.

In step S506, the control unit 3 detects the difference in air pressure between the outside and the air channel 10E through the pressure sensor 8. The control unit 3 can detect the start of inhalation by the user when the air-pressure difference reaches a predetermined value.

In step S507, a predetermined amount (20 μL) of liquid medicine is discharged for a predetermined time (1 second). In step S508, the control unit 3 displays the message “STORE MOUTHPIECE” on the display portion 4 for a predetermined time.

By storing the mouthpiece 10H, the discharge head 10C changes from an open (dischargeable) state with respect to the air channel 10E, as shown with a dotted line in FIG. 9A, to a closed state in which the discharge head 10C is covered by the cover 21, as shown with a solid line. As shown in FIG. 9B, the holes for spatially connecting the discharge head 10C, the pressure sensor 8, and the pressurizing pump 11 to the air channel 10E become sealed within a common space by the cover 21.

In step S509, the control unit 3 controls the pressurizing pump 11 while determining the value detected by the pressure sensor 8, so as to withdraw the liquid medicine from the discharge head 10C to the liquid medicine tank 10A through the liquid medicine channel 10B. Because the pressure sensor 8, the pressurizing pump 11, and the discharge head 10C are covered with the cover 21, the pressure applied to the discharge head 10C increases. On the other hand, the liquid medicine tank 10A has the rubber stopper 10J attached thereto, which is configured to move within the liquid medicine tank 10A when the difference in pressure between the air outside the rubber stopper 10J and the liquid medicine in the liquid medicine tank 10A becomes 0.8 kPa or higher. Therefore, as the pressurizing pump 11 monitors the pressure sensor 8 and keeps the difference in air pressure between the interior of the cover 21 and the outside at 0.8 kPa or higher for a predetermined time, the rubber stopper 10J moves by a distance corresponding to an amount of liquid medicine withdrawn from the discharge head 10C to the liquid medicine tank 10A. In this manner, the liquid medicine remaining in the discharge head 10C can be returned to the liquid medicine tank 10A after each use, whereby the liquid chambers in the discharge head 10C can be kept free of liquid medicine when the liquid medicine is in a preserved state. Finally, in step S510, the control unit 3 turns off the power of the inhaler device 1 so as to end the operation.

While the present invention has been described with reference to exemplary embodiments, it is to be understood that the invention is not limited to the disclosed exemplary embodiments. The scope of the following claims is to be accorded the broadest interpretation so as to encompass all modifications and equivalent structures and functions.

This application claims the benefit of Japanese Application No. 2007-216874, filed Aug. 23, 2007, which is hereby incorporated by reference herein in its entirety.

What is claimed is:

1. An inhaler device comprising:
a medicine container configured to contain medicine;
a medicine discharger configured to discharge the medicine from the medicine container; and
a pressurizing unit configured to increase a pressure applied to the medicine discharger so as to return the medicine in the medicine discharger to the medicine container.

2. The inhaler device according to claim 1, further comprising:
a mouthpiece through which a user inhales the medicine; and
an air channel configured to guide the medicine discharged from the medicine discharger towards the mouthpiece in response to inhalation by the user,
wherein the medicine discharger is disposed facing the air channel, and
wherein the pressurizing unit is configured to increase pressure in the air channel.

3. The inhaler device according to claim 2, wherein the pressurizing unit includes:
the mouthpiece configured to be storable in the air channel; and
a mouthpiece cap configured to block the mouthpiece,
wherein the pressure in the air channel is increased by storing the mouthpiece in the air channel such that the mouthpiece is blocked by the mouthpiece cap.

4. The inhaler device according to claim 2, wherein the pressurizing unit includes a pressurizing pump configured to increase the pressure in the air channel.

5. The inhaler device according to claim 1, wherein the pressurizing unit includes:
a cover configured to seal the medicine discharger; and
a pressurizing pump configured to increase air pressure in the cover.

6. The inhaler device according to claim 1, wherein the medicine discharger includes an electrothermal transducer configured to apply thermal energy to the medicine or an electromechanical transducer configured to apply mechanical energy to the medicine.
